(19) United States
(12) Patent Application Publication
(10) Pub. No.: US 2009/0114848 A1
(43) Pub. Date: May 7, 2009

(54) CLUSTER FILM FORMATION SYSTEM AND FILM FORMATION METHOD, AND CLUSTER FORMATION SYSTEM AND FORMATION METHOD

(75) Inventors: Yasushi Iwata, Nagano (JP); Toshio Takiya, Osaka (JP)

Correspondence Address: YOUNG & BASILE, P.C., 3001 WEST BIG BEAVER ROAD, SUITE 624, TROY, MI, 48084, US

(73) Assignee: National Institute of Advanced Industrial Science and Technology, Tokyo (JP)

(21) Appl. No.: 12/096,703

(22) PCT Filed: Dec. 7, 2006
(86) PCT No.: PCT/JP2006/324459

§ 371 Date: Jun. 9, 2008

(30) Foreign Application Priority Data
Dec. 13, 2005 (JP) 2005-358927

(51) Int. Cl. A61N 5/00

(52) U.S. Cl. 250/492.1

(57) ABSTRACT

The invention provides a cluster film formation system in which, in a cluster formation container 5, target 1 is irradiated with laser beams 2 to generate material vapor, which generates a shock wave 4 of an inert gas, and the shock wave 4 is reflected by a wall of the cluster formation container 5 to confine the material vapor having progressed in a particular region, and atoms or molecules of the material vapor and the inert gas collide with each other mutually to form groups of clusters, which are made to flow out through an outflow window 7, and sprayed deposited on a substrate 9 to form a cluster film 10. Corresponding to augmentation of energy strength of the laser beams 2, a cross section area of the laser beams 2 on the surface of the target is made large, thereby an increase in the amount of generation of the material vapor and efficient generation of the shock wave of the inert gas both are realized, and at the same time, the cluster formation container is enlarged so that the reflected wave of the shock wave meets conditions for confining the material vapor.

### TECHNICAL FIELD

The present invention relates to a cluster film formation system and a film formation method, and a cluster formation system and a formation method by laser ablation used for depositing clusters on a substrate to form a film-like aggregate of clusters.

### BACKGROUND ART

In these years, it has been required to control characteristics of a microstructure equal to or smaller than 10 nm. It is because miniaturization is expected to cause material properties to change, providing application to many fields such as nanoelectronics, optical electronics, and biotechnologies. For a general film formation technique of material, Plasma Chemical Vapor Deposition (CVD), Ion Sputtering CVD, and Laser CVD have been conventionally used, and widely put to practical use in the industrial world because of their characteristic of efficient film formation capability on a large area substrate (Patent Document 1). However, as film formation techniques including control of a nanoscale microstructure required for a need for miniaturization reaching the nanoscale which has been recently increased, these CVD techniques inherently have a difficult, technical issue.

A representative approach to finely controlling a nanoscale microstructure using CVDs includes: an approach in which attached atoms gather together along crystal lattices on a surface of a substrate into an island-shaped periodic structure to epitaxially grow (Patent Document 2); and an approach in which semiconductor atoms are vapor-deposited on an amorphous structure substrate such as of SiO₂ using a low pressure CVD, or in which semiconductor atoms are injected into an SiO₂ thin film, and subsequently semiconductor nanocrystals are formed by using annealing at a high temperature (Non-patent Document 1). The former approach has aspects difficult as industrial techniques, the aspects being: sensitively depending on a state of the surface of a substrate such as cleanness, temperatures, flatness in the atomic level of the surface of the substrate, the fact that, because of also dependence on a deposition rate, nano-structure control is governed by the film formation rate; and a drawback that because the number of island-shaped periodic structures formed is limited to one layer, a multilayer nano-structure thin film cannot be created. The latter approach has many issues as industrial techniques for finely controlling a nano-structure, the issues being: sensitively depending on temperature control of a thin film substrate; a necessity of a multistep film formation process requiring, for example, annealing in a gas environment at a high temperature equal to or higher than 1000°; sensitively affecting in size distribution of formed nano-particles to the multistep deposition process; problematic occurrence of impurities in a vapor-deposition process of semiconductor atoms; impossible creation of a multilayer nano-structure thin film on the surface of a substrate, and the like.

Regarding a miniaturization control technique by nano-particle formation on a substrate using CVD techniques, a deposition technique for depositing a cluster (nano-particles) formed in the gas phase, as described in Patent Document 3, has been tested on application to a semiconductor device. However, this technique has drawbacks that, because it is difficult to control cluster dimensions, it has not been achieved to control properties of material by miniaturization, and because cluster formation and cluster deposition on a substrate are performed in the same, competing container similarly to the case of a CVD technique, the problem of occurrence of impurities in the deposition process has not been resolved, and a cluster mixes in an insulating film to form a pattern, so that density of clusters cannot be increased.

In addition, the term “cluster”, as herein used, describes an aggregate of atoms or molecules, and is here viewed as the synonymous term as nano-particles or nano-crystals.

In contrast, a cluster beam method has been utilized that a cluster formation process is performed in a container different from a vacuum container for a cluster deposition process, and the formed cluster is taken out as a beam. The cluster beam method includes: an approach in which a cluster is formed as ions, and caused to collide with a substrate by accelerating it up to a high speed to be dissociated into an atomic state, and subsequently a uniform atomic layer is formed; and an approach in which groups of electrically neutral clusters are made attached onto a substrate, and the clusters are deposited to form a cluster layer. Regarding the former approach, one example is shown in Patent Document 4, and only cluster ions formed from a gaseous base material has been utilized in a practical use, providing various practical products such as super-planarization of a surface for a substrate, creation of an ultra compact semiconductor thin film, and the like

On the other hand, it may be believed that the latter approach is suitable for a film formation technique for finely controlling a nano-structure which is the technical issue because groups of neutral clusters are deposited on a substrate to form a nano-structure for each cluster on the substrate. The neutral cluster beam method has advantages such as capability of forming a film having a high purity by attaching clusters onto a substrate placed in a separate, high-vacuum container separated from a cluster formation container through a microbore because of a high directivity of a cluster particle flow, and capability of forming a uniform film by scanning an attachment region defined definitely. In film formation in which a nano-structure is finely controlled, it becomes necessary to further control grain size of clusters, to enhance efficiency of film formation on a large area substrate which is a superior feature of CVD techniques in order to use as an alternative technique of the CVD techniques, and to augment a cluster beam to allow for a practical film formation.

Regarding uniform grain size control of clusters, a cluster formation method and a system thereof is described in Patent Document 5 are proposed. FIG. 9 shows operation principle of this improved system. First, a target material 1 placed at a point A is irradiated with laser beams 2 to generate vapor 3 of material atoms. Pressure of the material vapor impacts on an inert gas, for example, a He gas present in the front of it to create a shock wave 4. The shock wave 4 is reflected by a wall of a cluster formation container 5, and concentrates to make a focal point in a region B. At this time, the vapor 3 of the material atoms reaches just the region B, and confined by the inert gas which has been reflected to gather together, so that the material atoms are bound together to form a cluster 6. The cluster 6 is made to flow out from the formation container 5 through a window 7 of the container, to pass through a skimmer 8, and to perpendicularly collide with a substrate 9 to form a cluster film 10. A possibility of forming a film configured by clusters uniform in size to the extent of several nm by this method has been experimentally confirmed in Non-patent Document 2.

In application of the neutral cluster beam method to products, a manufacturing cost of a film is considered to be most important. It is then desirable that a total area of a film manufactured per unit time be is as large as possible. Further, as seen from the case of the LSI manufacturing, it may be often required that the film be adapted to a large area in a case such as where, to decrease a cost of products to which the film is applied, a substrate is made larger to increase efficiency in mass production. It, then, becomes necessary to increase the amount of cluster formation per unit time, and to allow a film formation rate to improve. Accordingly, development of a cluster beam film formation system has been long-awaited that uses a new technique for achieving augmentation of high-precision cluster beams which allow for a practical film formation, and are controlled in size.

[Patent Document 1] JP 2000-269146 A

[Patent Document 2] JP 09-092879 A

[Patent Document 3] JP 2004-134796 A

[Patent Document 4] JP 2004-063819 A

[Patent Document 5] JP 2001-158956 A

[Non-patent Document 1] B. Garrido Fernandez, et al., “Influence of average size and interface passivation on the spectral emission of Sᵢ nanocrystals embedded in SᵢO₂”, J, Appl. Phys., Vol. 91, No. 2, p 798 (2002)

[Non-patent Document 2] “Array order formation of silicon nano-block and practice of thin film formation system”, Japan Laser Processing Society, Journal, Vol. 10, No. 3, December 2003

### DISCLOSURE OF THE INVENTION

However, the techniques, as described in the above Patent Documents, have a very slow rate of film formation, and it then is essential to improve the techniques in productivity to a large extent for producing a lot of products to which this film is applied, even if the systems may be used for experiments and studies. That is, it is necessary to largely increase the amount of cluster production in the above improved systems while keeping uniformity in size of formed clusters which is a feature of the systems.

Further, to address the challenge of the increase in amount of cluster production, a means of augmenting irradiation strength of laser beams may be thought of for increasing the amount of evaporation of material vapor. In this case, it is necessary to efficiently generate a shock wave corresponding to the increase in the amount of vapor, and there is an issue that the generated shock wave is reflected by a wall of the cluster formation container to form a region for effectively confining the vapor.

Further, there are issues how to address a number of problems caused by the augmentation of the laser beam strength, that is, treatment for heat generation caused by introducing laser beams in a cluster film manufacturing system, measurement of strength distribution of the beams on a target irradiation surface, and prevention from shrinkage of the target surface caused by evaporation.

The present invention has been made under the circumstances described above, and an object thereof is to provide a cluster film formation system and a film formation method, and a cluster formation system and a formation method which achieve an improved formation rate of a cluster film by providing a means for effectively forming groups of clusters using laser beams whose strength is augmented for increasing cluster production.

Further, an object of the present invention, solving the many problems caused by the augmentation of the laser beam strength for increasing the amount of evaporation of material vapor, is to provide a cluster film formation system and a film formation method, and a cluster formation system and a formation method for mass production which can increase the amount of generation of material vapor by augmenting the laser beam strength, and form a large volume of clusters.

To solve the above problems, a cluster film formation system according to claim 1 includes: a cluster formation container in which a target material providing raw material of clusters is placed at a predetermined position, and groups of clusters are formed while an inert gas is introduced; a laser beam source which irradiates the target material with laser beams from the outside of the cluster formation container; and a cluster film formation container in communication with the cluster formation container, for forming a cluster film on a predetermined substrate, in which material vapor of the target material irradiated with the laser beams generates a shock wave of the inert gas, the shock wave is reflected by an inner wall of the cluster formation container to confine the material vapor in a particular region, atoms or molecules of the material vapor and the inert gas collide with each other mutually to form the groups of clusters of the material, the groups of clusters are made to flow out through an outflow window provided in a wall of the cluster formation container in the extended line of a straight line connecting the target material and the particular region toward a substrate placed in the cluster film formation container in a vacuum state, the groups of clusters are changed into cluster beams by enhancing directivity of the flow of the groups of clusters through a skimmer toward the substrate, and the groups of clusters are deposited on the substrate in the cluster film formation container to form a cluster film, the cluster film formation system being characterized by including an energy density setting means for setting energy strength of the laser beams to equal to or greater than 300 mJ, and setting density of the energy so as to fall within a predetermined range on the target material, in which a distance from an irradiation surface of the target material to the outflow window is set to ten or more times larger than a beam size on the surface of the target material.

The beam energy strength is 50 to 300 mJ in the example of Non-patent Document 2, and it is necessary to augment the strength to a large extent compared to this. In this case, in order to avoid occurrence of a situation that, because of a high energy supplied in a concentrated manner to a material target, the material partially melts instantaneously, and the target material, remaining in the form of liquid, flies apart without turning into vapor to generate splashes, and the target material then is excessively consumed to lower efficiency of vapor creation, it, then, becomes necessary to make a beam cross section area on the target surface large to set the irradiation beam energy density to equal to or smaller than a limit value. An average irradiation beam energy density equal to or smaller than 100 mJ/mm² has been experimentally confirmed, but the density greater than 1000 mJ/mm² may be thought to be possibly problematic.

Here, the energy density to be set by enlargement of the beam cross section area on the target surface is provided by placing the target surface at a point shifted from a convergent point of the laser beams condensing at a small angle.

Further, laser strength distribution of the beam cross section on the target surface affects density distribution of generated vapor, and depends on shock wave generation of an inert gas, and adjustment is necessary so as to optimize the shock wave generation efficiency of the inert gas. In addition, the shock wave generation has an optimum point from the relation between particle density of the inert gas in a container and pressure of the material vapor.

When the beam cross section area is made large in such a manner, conditions are set as follows under which the material vapor is confined by a shock wave reflected by a wall of the container. For example, in the case shown in FIG. 9, in order to confine the material vapor in the region B by the reflected wave, it is required that a wall surface of the container forms in an ellipsoid of revolution, and the target and the region B are positioned at two focal points of the ellipsoid of revolution, respectively. When a beam cross section area at the target position can be viewed as a point, a shock wave spherically broadens from an originating point of the shock wave, and is reflected by the wall of the container to converge on the region B. However, when the beam cross section area is made large as described above, the reflected wave will not converge on the focal point to form a confining region if the same container is used. That is, the shock wave emitted from a beam irradiation surface will not form in a spherical shape. However, as away from the beam irradiation surface, broadening of the shock wave forms in a shape closer to a spherical shape, and can be approximately viewed as a spherical shape, for example, at a position far away by ten or more times larger than a diameter of the beam irradiation surface. Then, when the beam cross section area is enlarged and has particular dimensions, the length of a long axis of the container is set to ten or more times longer than the diameter of the beam cross section, which can provide a region in which the shock wave of the inert gas induced by the material vapor generated from the target surface is reflected by the wall of the container, and approximately converges, that is, the region B.

Further, the container is correspondingly enlarged in the short axis direction, and the value is set corresponding to a distance between a position of the convergent point B of the reflected wave and a position of the window through which clusters flow out from the container.

In addition, involving the enlargement of the container, dimensions of the cluster outflow window are made large; thereby efficiency of the cluster outflow can be enhanced.

As described above, owing to the augmentation of the laser beam energy strength, the increased amount of vapor generation caused by the enlargement of the area for generating vapor on the target surface, and the setting so as to meet dimensional conditions of the cluster formation container, the cluster film formation system according to claim 1 is intended to increase the amount of cluster production and improve the formation rate of a cluster film.

In addition, in the above description, the wall surface of the container has been formed in the ellipsoid of revolution, but the wall surface may not be partially formed in the ellipsoid of revolution as long as an equivalent reflected wave may be formed.

Further, the present invention according to claim 2 relates to the cluster film formation system according to claim 1, and is characterized by including, to introduce the laser beams, an entrance window provided at a position different from that of the outflow window in the cluster formation container, and opened for allowing the laser beams to pass through.

In this configuration, an irradiation angle of the laser beams onto the target surface is set in the direction shifted from a traveling direction of generated vapor, that is, a direction toward the confining region of vapor. Then, a position of a substrate for cluster film formation and a light path of the laser beams can be set not to overlap with each other. Further, because the entrance window for introducing the laser beams into the cluster formation container is not sealed with material such as an optically transparent plate material, problems can be avoided that the sealing material is destroyed due to large energy strength of the laser beams, and reflected waves of the beams are generated. Further, the entrance window is provided at, or near the convergent point of the laser beams, and has very small dimensions, which can also reduce the amount of outflow of the inert gas in the cluster formation container.

Further, the present invention according to claim 3 relates to the cluster film formation system according to claim 1, includes an external container for containing the cluster formation container in a vacuum or quasi-vacuum environment, and is characterized in that the external container has an extension formed by extending an outline thereof in a tubular shape for passing through the laser beams, the extension has a sealing window on the side from which the laser beams are introduced, and in the sealing window, a plate material processed for antireflection of the laser beams is provided, and the sealing window is provided spaced away from the cluster formation container by a predetermined distance.

In this configuration, the structure of the window provided in the external container is proposed to introduce intensive laser beams from the outside of the external container surrounding the outside of the cluster formation container. The external container is set to be in a vacuum or quasi-vacuum environment, and the window is a window sealed with an optically transparent material to keep airtightness. To pass through the intensive laser beams, first, the window is placed at a position spaced far away from a bore of a window in the cluster formation container, the bore being set to be a focal point of the laser beams, thereby the cross section area through which the beams pass is enlarged to reduce energy density of the beams. In addition, here, settings are configured so that the laser beams are made to concentrate at a small angle, and after concentrating at the position of the bore of the window in the cluster formation container, the laser beams have a desired irradiation area and strength distribution at a position of the target material in the cluster formation container.

Further, when the laser beams pass through the sealing window, the laser beams passing through may be attenuated, and reflected beams may return back to the laser beam source to destroy the device if the laser beams are reflected by the surface and the underside surface of the optically transparent material. Then, to prevent reflection, the both surfaces of the optically transparent material are polished to be flat, and processed for antireflection such as application of an antireflection coating.

Further, the present invention according to claim 4 relates to the cluster film formation system according to claim 3, and is characterized in that the cross section area of the laser beams which pass through the sealing window of the external container is made large to such an extent that the energy density is lowered enough not to damage the sealing window, and the sealing window is tilted to make a predetermined angle with the surface perpendicular to an incoming optical axis of the laser beams so that reflection of the laser beams will not return back to the laser beam source.

In this configuration, the structure of the sealing window provided in the external container is proposed to introduce the intensive laser beams from the outside of the external container surrounding the outside of the cluster formation container, and this configuration is characterized in that the sealing window, similarly to claim 3, is placed away from the bore of the window in the cluster formation container by a predetermined distance, and the optically transparent material is mounted on the sealing window in a manner that the surface of the optically transparent material is shifted from the surface perpendicular to the optical axis of the laser beams. Consequently, reflected laser beams by the surfaces of the optically transparent material are caused not to return back in the direction toward the incoming optical axis of the laser beams.

Further, the present invention according to claim 5 relates to the cluster film formation system according to claim 3, and is characterized in that the sealing window of the external container, to introduce the laser beams, is placed in the extended line of a straight line connecting the entrance window provided in the cluster formation container and the target material.

Conventionally, to make systems small, laser beams have been reflected by a mirror disposed at a middle between an entrance window of an external container and an entrance window of a cluster formation container. However, in this configuration, the mirror may be omitted, and the laser beams may be provided in straight lines to a target surface through the sealing window of the external container, and an optical control system, therefore, is simplified to allow for a precise optical control.

Further, the present invention according to claim 6 relates to the cluster film formation system according to claim 1, and is characterized by including a laser beam condenser lens for condensing the laser beams, provided outside of the sealing window of the external container and the external container, and a mirror for changing the direction of all or a part of the strength of the laser beams, disposed in the optical axis between the laser beam condenser lenses for condensing the laser beams, in which the mirror is disposed so that the laser beams whose direction has been changed and the laser beams progressing onto the surface of the target material in the cluster formation container both have a like form in light condensing.

This configuration allows the mirror for changing the direction of all or a part of the energy of the laser beams to be inserted in the optical axis between the sealing window of the external container and the laser beam condenser lens disposed outside of the external container, and the laser beams whose direction has been changed outside of the external container re-creates similar characteristics of the laser beams on the target surface in the cluster formation container. Consequently, the strength and strength distribution of the beams on the target surface can be estimated outside of the external container to allow for a beam strength control for optimizing the generation efficiency of material vapor on the target surface.

Further, the present invention according to claim 7 relates to the cluster film formation system according to claim 1, and is characterized by including a support device for supporting the target material, in which the support device has: a function for moving the laser irradiation position on the surface of the target material by rotating the target material; and a function for pushing out the target material in the direction perpendicular to the surface by the amount corresponding to shrinkage caused by evaporation of the target material on the surface due to laser irradiation, and keeping the position of the irradiation surface to be constant.

This configuration gives the device for supporting the target according to claim 1; the function for moving the laser irradiation position on the target surface by rotating the target; and the function for pushing out the target in the direction perpendicular to the surface by the amount corresponding to the shrinkage caused by evaporation on the surface due to laser irradiation, and keeping the position of the irradiation surface to be constant.

For example, by rotating the disc-like target, the position on the surface is shifted for each irradiation of pulsed laser beams to equalize the shrinkage of the material on the surface caused by evaporation, and the target is pushed out in the direction toward the surface to compensate the shrunk portion on the surface at every moment, and at the same position of the surface, irradiation of the laser beams is applied. Consequently, the relation between the beam irradiation position in the cluster formation container and the position of the cluster outflow window is kept constant, and situations for cluster formation can be maintained to be constant.

Further, the cluster formation system according to claim 8 includes: a cluster formation container in which target material providing raw material of clusters is placed at a predetermined position, and groups of clusters are formed while an inert gas is introduced; and a laser beam source which irradiates the target material with laser beams from the outside of the cluster formation container, in which material vapor of the target material irradiated with the laser beams generates a shock wave of the inert gas, the shock wave is reflected by an inner wall of the cluster formation container to confine the material vapor in a particular region, atoms or molecules of the material vapor and the inert gas collide with each other mutually to form the groups of clusters of the material, and the groups of clusters are made to flow out through an outflow window provided in a wall of the cluster formation container in the extended line of a straight line connecting the target material and the particular region, the cluster formation system being characterized by including an energy density setting means for setting energy strength of the laser beams to equal to or greater than 300 mJ, and setting density of the energy so as to fall within a predetermined range on the target material, in which a distance from the irradiation surface of the target material to the outflow window is set to ten or more times larger than a beam size on the surface of the target material.

This configuration, not limited to the cluster film formation system according to claim 1, provides the cluster formation system, and allows for economical cluster formation by widely improving a cluster manufacturing capability of the conventional system.

Further, the cluster film formation method according to claim 9, in a cluster formation container filled with an inert gas includes: irradiating a target material providing raw material of clusters with laser beams; generating a shock wave of the inert gas by using generated material vapor; confining the material vapor in a particular region by using the shock wave reflected by a wall of the cluster formation container; forming groups of clusters of the material by using atoms or molecules of the material vapor and the inert gas colliding with each other mutually; making the groups of clusters which flow out through a window provided in a wall of the cluster formation container in the extended line of a straight line connecting the target material and the particular region; and depositing the groups of clusters on a predetermined substrate to form a cluster film, the method being characterized by including an energy density setting means for setting energy strength of the laser beams to equal to or greater than 300 mJ, and setting density of the energy so as to fall within a predetermined range on the target material, in which a distance from the irradiation surface of the target material to the outflow window is set to ten or more times larger than a beam size on the surface of the target material.

According to this configuration, similarly to claim 1, by augmenting the energy strength of the laser beams, increasing the amount of vapor generation caused by enlarging the vapor generation area on the target surface, and setting so as to meet the conditions for dimensions of the cluster formation container, the amount of cluster production can be increased to improve a formation rate of a cluster film.

Further, the cluster formation method according to claim 10, in a cluster formation container filled with an inert gas includes: irradiating a target material providing raw material of clusters with laser beams; generating a shock wave of the inert gas by using generated material vapor; confining the material vapor in a particular region by using the shock wave reflected by a wall of the cluster formation container; forming groups of clusters of the material by using atoms or molecules of the material vapor and the inert gas colliding with each other mutually; and making the groups of clusters flow out through a window provided in a wall of the cluster formation container in the extended line of a straight line connecting the target material and the particular region, the method being characterized by including an energy density setting means for setting energy strength of the laser beams to equal to or greater than 300 mJ, and setting density of the energy so as to fall within a predetermined range on the target material, in which a distance from the irradiation surface of the target material to the outflow window is set to ten or more times larger than a beam size on the surface of the target material, which increases the amount of formation of the groups of clusters and the amount of the groups of clusters taken out from the cluster formation container.

This configuration, similarly to claim 8, not limited to the cluster film formation system according to claim 1, provides the cluster formation method, and allows for economical cluster formation by widely improving a cluster manufacturing capability by the conventional method.

The configurations of the present invention, to enhance a film manufacturing rate of nano-clusters uniform in size, provide effective, mass production of clusters by optimally setting the increased beam energy of the laser beams, the beam size for target irradiation, and the dimensions of the cluster formation container, and further solve a number of the problems caused by the augmentation of the laser beam strength to increase the amount of evaporation of the material vapor, and address rapid shrinkage of the target material at this time to allow for constant cluster formation. Consequently, the cluster film formation techniques and the systems thereof can be provided that allow for economical efficiency required for cluster film formation.

### BRIEF DESCRIPTION OF THE DRAWINGS

FIG. 1 is a schematic diagram showing an overall configuration of a cluster film formation system according to a first embodiment of the present invention;

FIG. 2 is a schematic diagram illustrating a cluster formation mechanism in a cluster formation container of the cluster film formation system according to the first embodiment of the present invention, and a configuration of the cluster formation container involving an increase in energy of laser beams;

FIG. 3 is a view illustrating conditions for the relation between “d” and “x” in FIG. 2;

FIG. 4 is a view illustrating conditions for the relation between “d” and “x” in FIG. 2;

FIG. 5 is a schematic diagram showing a configuration of a laser beam introducing portion according to a second embodiment of the present invention;

FIG. 6 is a schematic diagram illustrating a mounting angle of an optically transparent material for sealing a window of an external container introducing laser beams according to a third embodiment of the present invention;

FIG. 7 is a schematic diagram of a system for evaluating characteristics of a laser beam system according to a fourth embodiment of the present invention, that is, a laser beam strength distribution on a target surface;

FIG. 8 is a schematic diagram illustrating situations of vapor generation of the target material when the laser beams are provided onto the target in a region of a target irradiation position in the cluster formation container according to a fifth embodiment of the present invention; and

FIG. 9 is a schematic diagram showing operation principle of a conventional example.

### DESCRIPTION OF SYMBOLS

target material
laser beam
2′ laser beam whose direction is changed
material vapor
shock wave
cluster formation container
group of cluster
outflow window
skimmer
substrate
cluster film
external container
11′ extension
sealing window (optically transparent material)
bore of window
13′ focal point
cluster film formation container
target irradiation surface
target irradiation position
19′ position equivalent to target irradiation position
measurement device of laser beam strength distribution
ND filter
support device
inert gas reservoir
inert gas inlet
phasic static flow of inert gas
inert gas flow ejecting from outlet of cluster formation cell
skimmer
cluster beam
reflected laser beam
R rotation direction
T push direction

### BEST MODE FOR CARRYING OUT THE INVENTION

A cluster film formation system according to embodiments of the present invention will be hereinafter described with reference to the accompanying drawings.

### First Embodiment

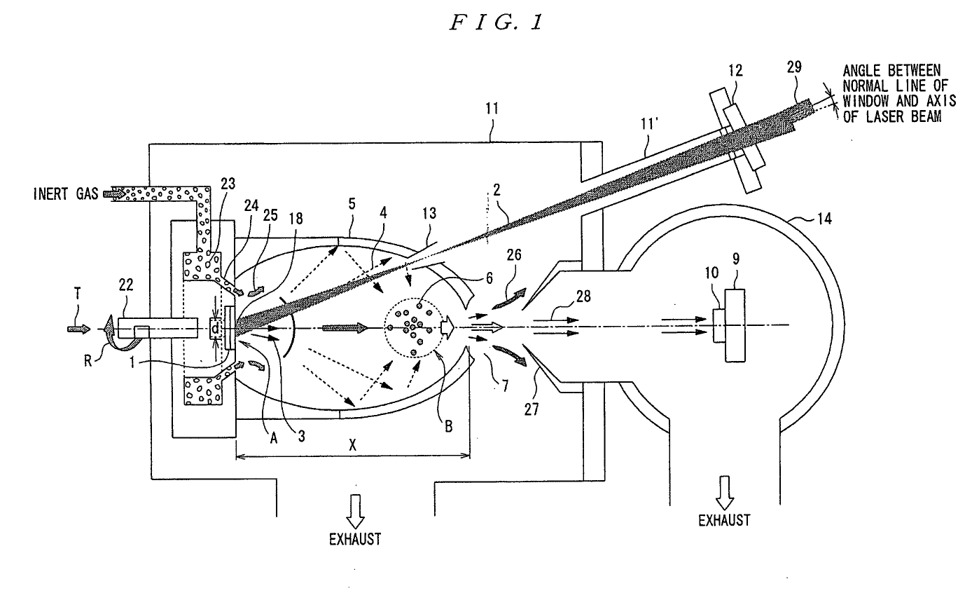

First, referring to FIG. 1, a first embodiment of the present invention will be described.

FIG. 1 is a schematic diagram showing an overall configuration of a cluster film formation system according to the first embodiment of the present invention.

This cluster film formation system includes a cluster formation container 5 for forming groups of clusters 6, a laser beam source for providing laser beams 2 (not shown), and a cluster film formation container 14 in which a substrate 9 having the groups of clusters 6 sprayed deposited thereon is placed.

The cluster formation container 5 has target material 1 providing raw material of cluster placed therein, and forms groups of clusters while introducing an inert gas. Further, the cluster formation container 5 has an outflow window 7 for making the groups of clusters flow out, and an entrance window 13 for introducing the laser beams 2, provided at a position different from that of the outflow window 7, and the entrance window 13 is opened.

The laser beam source provides the laser beams 2 onto the surface of the target material 1 from the outside of the cluster formation container 5. An irradiation surface on the target material 1 is shown by the reference number “18” in FIG. 1.

The cluster film formation container 14 communicates with the cluster formation container 5, and has a predetermined substrate 9 placed therein, and on the substrate 9, the groups of clusters 6 flowing out from the cluster formation container 5 are deposited to form a cluster film 10.

In this configuration, material vapor of the target material 1 irradiated with the laser beams 2 causes a shock wave 4 of the inert gas, the shock wave 4 is reflected by an inner wall of the cluster formation container 5 to confine the material vapor in a particular region B, atoms or molecules of the material vapor and the inert gas collide with each other mutually to form the groups of clusters 6 of the material, the groups of clusters 6 are made to flow out through the outflow window 7 provided in a wall of the cluster formation container 5 in the extended line of a straight line connecting the target material 1 and the particular region B, and the groups of clusters 10 are sprayed deposited on the substrate 9 in the cluster film formation container 14 to form the cluster film.

In this case, the present invention is characterized by including an energy density setting means for setting energy strength of the laser beams to equal to or greater than 300 mJ, and setting density of the energy so as to fall within a predetermined range on the target material, in which a distance from the irradiation surface of the target material to the outflow window is set to ten or more times larger than a beam size on the surface of the target material. The energy density setting means includes an overall configuration of an optical system for setting the energy density of the laser beams to a predetermined value on the target.

In addition, as shown in FIG. 1, the cluster formation container 5 has: an inert gas reservoir 23 provided on the side from which the inert gas is introduced, having a ring-shaped structure symmetrical with respect to a cell central axis; and an inert gas inlet 24 in communication with the inert gas reservoir 23, for forming an inert gas flow having a planar shape symmetrical with respect to an axis through a gap of the ring-shaped structure, in which the inert gas having passed through the inert gas inlet 24 becomes a phasic static flow without any turbulent flow to enter the cluster formation container 5. Consequently, a wave surface of vapor can be prevented from disturbance.

Further, when the inert gas flow is exhausted outside of the cluster formation container 5, after passing through the outflow window 7, it becomes an inert gas flow and exits outside. Then, a skimmer 27 is provided so that the central portion of the discharge jet of the inert gas can pass through to stop a spreading portion of the flow by applying an electric potential, thereby ion components are prevented from passing through, and as the result, to form a neutral beam.

The central portion of the discharge jet of the inert gas having passed through the skimmer 27 becomes cluster beams 28 to enter the cluster film formation container 14.

In addition, a window composed of an optically transparent material 12 is installed so that an axis of the laser beams 2 and a normal line of the window make a predetermined angle therebetween, and reflected laser beams 29 of the laser beams 2 and a normal line of the window make a predetermined angle therebetween, and reflected laser beams 29 of the laser beams 2 go out of the axis of the laser beams 2.

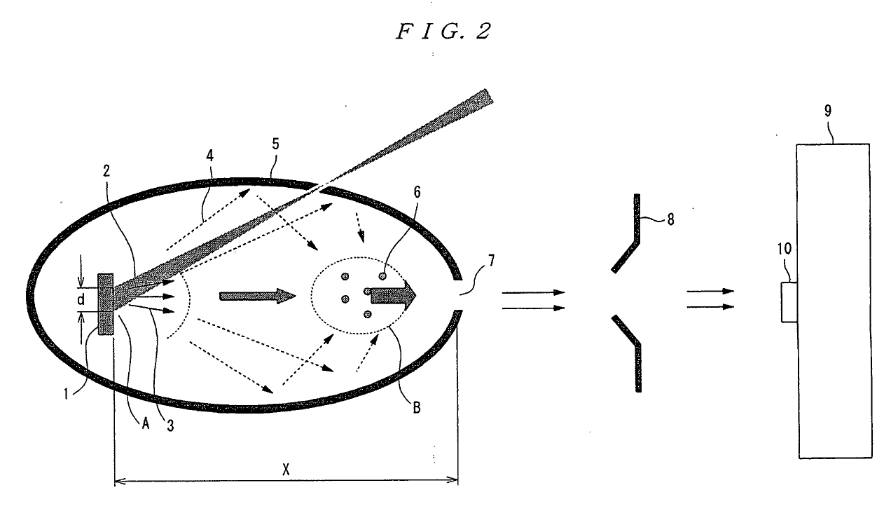

FIG. 2 is a schematic diagram illustrating a cluster formation mechanism in the cluster formation container in the cluster film formation system according to the first embodiment of the present invention, and a configuration of the cluster formation container involving an increase in energy of the laser beams.

In the cluster film formation system in the cluster formation container 5 filled with an inert gas including: irradiating target material 1 providing raw material of the clusters with laser beams 2; generating a shock wave 4 of the inert gas by using generated material vapor 3; confining the material vapor 3 having progressed in a particular region B by using the shock wave 4 reflected by an inner wall of the cluster formation container 5; forming groups of clusters 6 of the material by using atoms or molecules of the material vapor 3 and the inert gas 25 colliding with each other mutually; making the groups of clusters 6 flow out through a window 7 provided in a wall of the cluster formation container 5 in the extended line of a straight line connecting the target material 1 and the particular region B; making the groups of clusters 6 having flowed out pass through a skimmer 8 to be sprayed deposited on a substrate 9; and forming a cluster film 10, in order to increase the amount of cluster film production, first, strength of the laser beams 2 is enhanced, and a beam cross section area of surface irradiation on the target material 1 is enlarged, and at this time, laser strength distribution on the irradiation cross section is adjusted, thereby a large volume of the material vapor 3 and the shock wave 4 of the inert gas caused by the material vapor 3 are efficiently generated, and the shock wave reflected by a wall of the container 5 enlarged in dimensions confines the material vapor in the region B to form clusters. Here, when a diameter of the beam cross section of surface irradiation is “d”, a distance “x” from the target material 1 in the container 5 to the outlet 7 is made ten or more times greater than “d”, resulting in generation of efficiently confining situations at the region B, and thereby a massive amount of clusters can be formed from a large volume of the material vapor generated by the increased laser beam energy.

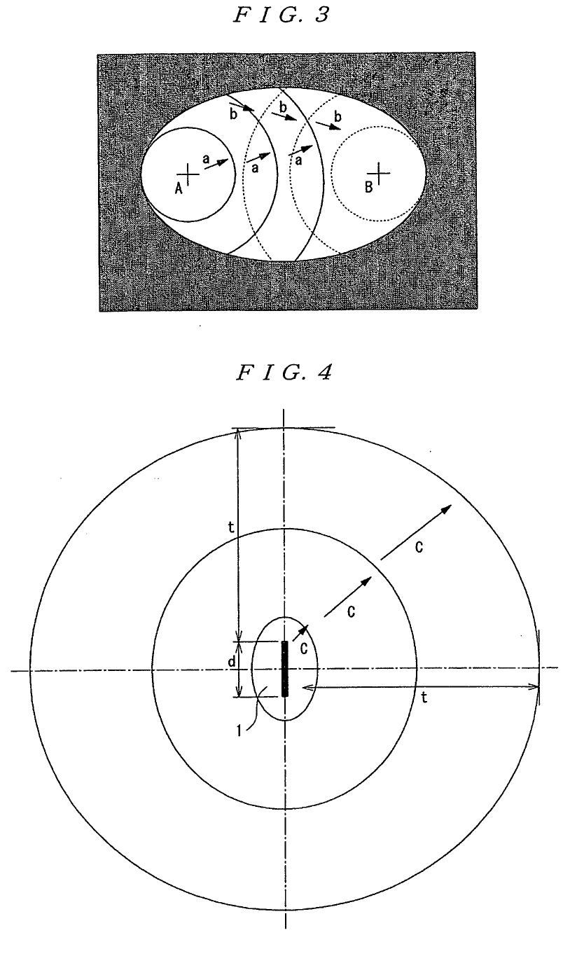

FIGS. 3 and 4 are views for illustrating conditions for the relation between “d” and “x” in FIG. 2.

FIG. 3 shows that, when the beam cross section area of irradiation on the target surface is small enough to be viewed as a point and the point is indicated by a point A, a shock wave generated at the point A, as shown the arrow “a”, broadens spherically, and is reflected by an inner wall of a container having an ellipsoidal shape of revolution, then as shown by the arrow “b”, converges spherically at a point B. That is, at the point B, a confining region by using the shock wave is formed. However, as shown in FIG. 4, when the beam cross section area of irradiation on the target surface has a finite value “d”, a shock wave generated at the irradiation surface will not be spherical. That is, when a wave surface of the shock wave progresses by a distance “t” in the direction perpendicular to the irradiation surface, the wave surface will position at “t+d/2” in the direction horizontal to the irradiation surface.

However, when the distance “t” is ten or more times larger than the dimension “d”, the distances to the wave surfaces in the perpendicular direction and in the horizontal direction may be viewed as approximately the same distance, and it may be thought that the shock wave broadens spherically. Then, by making a length of a long axis of the container having the ellipsoidal shape of revolution ten or more times larger than “d”, the condition is met and the confining region by the shock wave is provided effectively at the point B. In such a manner, a production capability of the cluster film formation system of the present invention can be considerably enhanced.

In addition, as shown in FIG. 2, an incoming direction of the laser beams 2 into the cluster formation container 5 is shifted from an axis connecting the target material 1 and the cluster outflow window 7 by a particular angle, and a window for allowing the laser beams 2 to enter the cluster formation container 5 is not sealed with an optically transparent material or the like, and opened.

### Second Embodiment

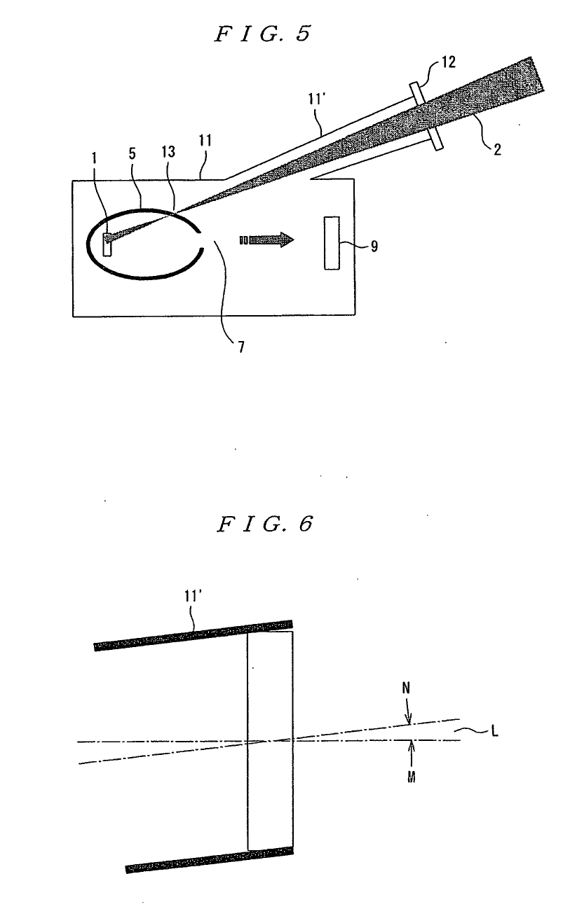

Next, referring to FIG. 5, a cluster film formation system according to a second embodiment of the present invention will be described.

FIG. 5 is a schematic diagram for illustrating a configuration of a laser beam introducing portion.

The second embodiment proposes a structure of a window provided in an external container 11 to introduce intensive laser beams 2 from the outside of the external container 11 surrounding the outside of a cluster formation container 5 as shown in FIG. 5. The external container 11 is brought into a vacuum or quasi-vacuum environment, and the window maintains airtightness with an optically transparent material 12. Then, to pass through the intensive laser beams 2, first, the window is positioned to lower energy density of the beams passing through the window by providing a predetermined distance from a position of a bore 13 of a window in the cluster formation container 5, the bore 13 of the window being positioned at a focal point of the laser beams 2. For the purpose, m this embodiment, the external container 11 is extended by adding a cylindrical tube shown by 11′ (hereinafter, called “extension”). In addition, a laser beam system is configured so that the laser beams 2 are focused at the position of the bore 13 of the window in the cluster formation container 5, and an irradiation area is enlarged on an irradiation surface of the target 1.

Further, when the laser beams 2 pass through the optically transparent material 12 in the extension 11′, the laser beams 2 passing through may be attenuated, and reflected beams may return back to the laser beam source to destroy the system if the laser beams are reflected by the surface and the underside surface of the optically transparent material. Then, to prevent the reflection of laser, the both surfaces of the optically transparent material 12 are polished to be flat, and processed for application of an antireflection coating.

In addition, as shown in FIG. 4, the optical axis of the laser beams 2 forms a straight line from the sealing window composed of the optically transparent material 12 in the external container 11 to the target material 1, and the optical axis is not deflected by a mirror or the like in the external container 11, so that the external container 11 is not made small. Consequently, the optical system can be more precisely controlled.

### Third Embodiment

Next, referring to FIG. 6, a cluster film formation system according to a third embodiment of the present invention will be described.

FIG. 6 is a schematic diagram illustrating a mounting angle of the sealing window 12 composed of the optically transparent material provided in the extension 11′ of the external container 11 for introducing the laser beams 2 shown in FIG. 5.

The third embodiment, as shown in FIG. 6, relates to a structure of the sealing window 12 (optically transparent material) in the extension 11′ formed by extending the external container 11 for introducing the intensive laser beams 2 from the outside of the external container 11 surrounding the outside of the cluster formation container 5. That is, the structure is characterized in that a perpendicular M to the surface of the sealing window 12 (optically transparent material) is shifted from an optical axis N of the laser beams 2 by a predetermined angle L when the sealing window 12 (optically transparent material) for sealing the window is installed. Consequently, reflected laser beams by the surfaces of the sealing window 12 (optically transparent material) will not return back in the direction of the optical axis N of the laser beams 2, and similarly to the second embodiment, the reflected beams can be prevent from returning back to the laser beam source to destroy it.

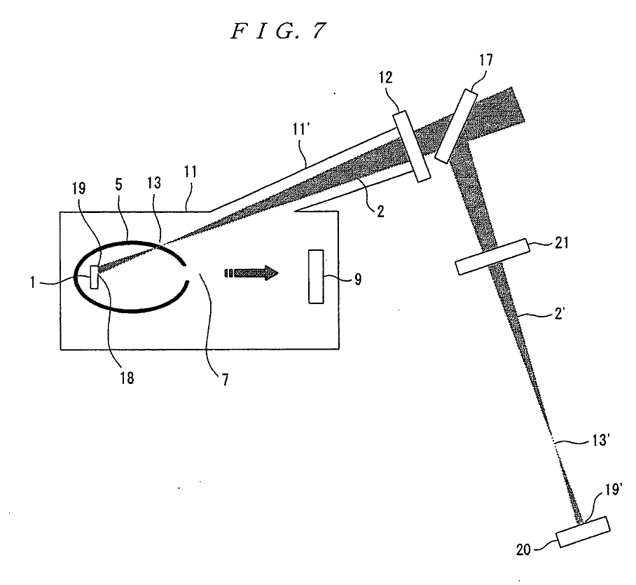

Next, referring to FIG. 7, a cluster film formation system according to a fourth embodiment of the present invention will be described.

FIG. 7 is a schematic diagram showing a configuration of a system for evaluating characteristics of the laser beam system, that is, a laser beam strength distribution on the surface of the target material 1.

The fourth embodiment, as shown in FIG. 7, allows the beam characteristics to be evaluated by inserting a mirror 17, to change the direction of all or a part (about 1%) of energy of the laser beams, in the optical axis of the laser beams 2 incident on the sealing window 12 of the external container 11 while smoothly condensing the laser beams 2 by using a laser beam condenser lens further outside of the extension 11′ of the external container 11 surrounding the outside of the cluster formation container 5. That is, deflected beams 2′ re-create similar situations to the beams progressing from a place at which the mirror 17 is inserted up to the irradiation surface 18 of the target material 1 in the cluster formation container 5. Correspondingly to the focal point of the laser beams 2 made at the position of the bore 13 through which the laser beams enter the cluster formation container 5, the deflected beams are also focused at a point 13′, and subsequently the strength distribution of the laser beams 2 can be estimated with a measurement device 20 of laser beam strength distribution disposed at a point 19′ equivalent to a target irradiation position 19. In addition, a neutral density (ND) filter 21 is inserted in the way of the deflected beams 2′ to attenuate the laser beams. The ND filter 21 uniformly absorbs any light wavelength. This configuration, outside of the external container 11, allows the situations of the laser beams 2 in the cluster formation container 5 to be understood and the system of the laser beam source to be adjusted for optimization.

### Fifth Embodiment

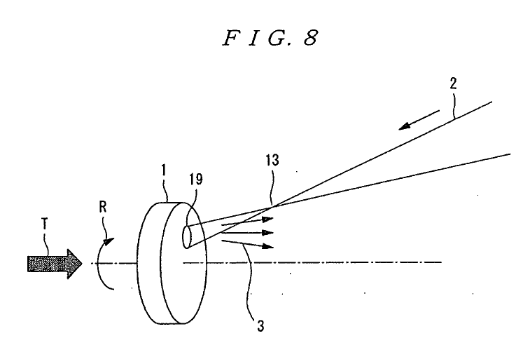

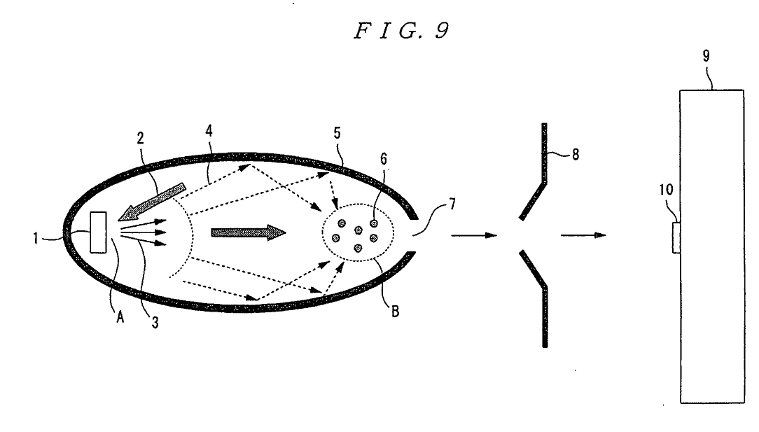

Next, referring to FIG. 8, a cluster film formation system according to a fifth embodiment of the present invention will be described.

FIG. 8 schematically illustrates situations of generating the material vapor 3 of the target material 1 when the target material 1 in the cluster formation container 5 is irradiated with the laser beams 2, in a region of the irradiation position 19 in the target material 1.

In the fifth embodiment, as shown in FIG. 8, by rotating the target material 1 in the direction shown by the arrow R, the irradiation position 19 of the laser beams is moved on the surface of the target material 1 to equalize the shrinkage of the target material 1 on the surface caused by evaporation. However, only by doing so, the position of the laser beam irradiation surface 18 will be shifted. Then, the support device 22 for supporting the target material 1 may have a function for rotation, and at same time, a function in which the target material 1 is pushed out in the direction approximately perpendicular to the surface of the target material 1 by the amount corresponding to the shrinkage caused by evaporation of the target material 1 on the surface as shown by the arrow T, and keeping the position of the irradiation surface to be constant. Consequently, the situations in the cluster formation container S are kept constant and a cluster formation state can be maintained to be constant.

While the embodiments of the present invention have been described above, the present invention is not limited to these embodiments, and various modifications may be made thereto without departing from the scope of the invention.

For example, in the embodiments described above, the support device 22 for supporting the target material 1 has been illustrated to perform the rotation movement shown by R and the horizontal movement shown by T, but not limited to those, the support device 22 may move the target material 1 in any direction such as an oblique direction, a vertical direction up and down, and an irregular movement to further enlarge the irradiation area of the laser beams.

Further, in the embodiments described above, a room for spraying depositing the cluster film 10 on the substrate has been the cluster film formation container 14, but not limited to this, the cluster film 10 may be formed in a vacuum chamber in a vacuum or quasi-vacuum environment similar to the external container 11.

Further, in the embodiments described above, the distance from the irradiation surface of the target material 1 to the outflow window 7 has been set to ten or more times larger than the maximum size of the irradiation area on the target material 1, but not limited to this, if the region B may be formed at the front of the outflow window by changing the shape of the cluster formation container 5, a similar effect may be provided.

Further, in the embodiments described above, there has been provided the example having the sealing window 12 composed of the optically transparent material placed on the side of the extension 11′ of the external device 11 from which the laser enters, but not limited to this, various material may be used as long as the material may pass through and not reflect laser beams.

### INDUSTRIAL APPLICABILITY

As described above, the present invention can provide, to increase the amount of cluster film production, the optimum cluster outflow window which realizes both of effective cluster formation by augmenting the beam strength of the laser beams and enlarging the dimensions of the cluster formation container, and efficient outflow of the groups of clusters formed from the cluster formation container, and further solves a number of the problems caused by the augmentation of the laser beam strength to increase the amount of evaporation of the material vapor, and allows for a constant cluster formation by addressing the rapid shrinkage of the target material on this occasion.

1. A cluster film formation system, comprising:
a cluster formation container in which a target material providing raw material of clusters is placed at a predetermined position, and groups of clusters are formed while an inert gas is introduced;
a laser beam source which irradiates the target material with laser beams from the outside of the cluster formation container; and
a cluster film formation container in communication with the cluster formation container, for forming a cluster film on a predetermined substrate, wherein
material vapor of the target material irradiated with the laser beams generates a shock wave of the inert gas,
the shock wave is reflected by an inner wall of the cluster formation container to confine the material vapor in a particular region,
atoms or molecules of the material vapor and the inert gas collide with each other mutually to form the groups of clusters of the material,
the groups of clusters are made to flow out through an outflow window provided in a wall of the cluster formation container in the extended line of a straight line connecting the target material and the particular region, and
the groups of clusters are deposited on the substrate in the cluster film formation container to form a cluster film, characterized by including:
an energy density setting means for setting energy strength of the laser beams to equal to or greater than 300 mJ, and setting density of the energy on the target material so as to fall within a predetermined range on the target material, wherein a distance from an irradiation surface of the target material to the outflow window is set to ten or more times larger than a beam size of the laser beams on the surface of the target material.

2. The cluster film formation system according to claim 1, characterized by further comprising:
to introduce the laser beams, an entrance window provided at a position different from that of the outflow window in the cluster formation container, and opened for allowing the laser beams to pass through.

3. The cluster film formation system according to claim 1, characterized by further comprising:
an external container for containing the cluster formation container in a vacuum or quasi-vacuum environment, wherein
the external container has an extension formed by extending an outline thereof in a tubular shape for passing through the laser beams,
the extension has a sealing window on the side from which the laser beams are introduced, and in the sealing window, an optically transparent plate material processed for antireflection of the laser beams is provided, and
the sealing window is provided spaced away from the cluster formation container by a predetermined distance.

4. The cluster film formation system according to claim 3, characterized in that
a cross section area of the laser beams which pass through the sealing window of the external container is made large to such an extent that energy density is lowered enough not to damage the sealing window, and
the sealing window is tilted to make a predetermined angle with the surface perpendicular to an incoming optical axis of the laser beams, so that reflection of the laser beams will not return back to the laser beam source.

5. The cluster film formation system according to claim 3, characterized in that
the sealing window of the external container, to introduce the laser beams, is placed in the extended line of a straight line connecting the entrance window provided in the cluster formation container and the target material.

6. The cluster film formation system according to claim 1, characterized by further comprising:
a laser beam condenser lens for condensing the laser beams, provided outside of the sealing window of the external container and the external container; and
a mirror for changing a direction of all or a part of the strength of the laser beams, disposed in an optical axis between the laser beam condenser lenses for condensing the laser beams, wherein
the mirror is disposed so that the laser beams whose direction has been changed and the laser beams progressing onto the surface of the target material in the cluster formation container both have like characteristics.

7. The cluster film formation system according to claim 1, characterized by further comprising:
a support device for supporting the target material, wherein
the support device has:
a function for moving a laser irradiation position on the surface of the target material by rotating the target material; and
a function for pushing out the target material in the direction perpendicular to the surface by the amount corresponding to shrinkage caused by evaporation of the target material on the surface due to laser irradiation, and keeping the position of the irradiation surface to be constant.

8. A cluster formation system, comprising:
a cluster formation container in which a target material providing raw material of clusters is placed at a predetermined position, and groups of clusters are formed while an inert gas is introduced; and
a laser beam source which irradiates the target material with laser beams from the outside of the cluster formation container; wherein
a material vapor of the target material irradiated with the laser beams generates a shock wave of the inert gas, p1 the shock wave is reflected by an inner wall of the cluster formation container to confine the material vapor in a particular region,
atoms or molecules of the material vapor and the inert gas collide with each other mutually to form the groups of clusters of the material, and
the groups of clusters are made to flow out through an outflow window provided in a wall of the cluster formation container in the extended line of a straight line connecting the target material and the particular region, characterized by comprising:
an energy density setting means for setting energy strength of the laser beams to equal to or greater than 300 mJ, and setting density of the energy so as to fall within a predetermined range on the target material, wherein
a distance from an irradiation surface of the target material to the outflow window is set to ten or more times larger than a beam size on the surface of the target material.

9. A cluster film formation method in a cluster formation container filled with an inert gas, comprising:
irradiating a target material providing raw material of clusters with laser beams;
generating a shock wave of the inert gas by using generated material vapor;
confining the material vapor in a particular region by using the shock wave reflected by a wall of the cluster formation container;
forming groups of clusters of the material by using atoms or molecules of the material vapor and the inert gas colliding with each other mutually;
making the groups of clusters flow out through a window provided in a wall of the cluster formation container in the extended line of a straight line connecting the target material and the particular region; and
depositing the groups of clusters on a predetermined substrate to form a cluster film, characterized by further comprising:
an energy density setting means for setting energy strength of the laser beams to equal to or greater than 300 mJ, and setting density of the energy so as to fall within a predetermined range on the target material, wherein
a distance from an irradiation surface of the target material to the outflow window is set to ten or more times larger than a beam size on the surface of the target material.

10. A cluster formation method in a cluster formation container filled with an inert gas, comprising:
irradiating a target material providing raw material of clusters with laser beams;
generating a shock wave of the inert gas by using generated material vapor;
confining the material vapor in a particular region by using the shock wave reflected by a wall of the cluster formation container;
forming groups of clusters of the material by using atoms or molecules of the material vapor and the inert gas colliding with each other mutually; and
making the groups of clusters flow out through a window provided in a wall of the cluster formation container in the extended line of a straight line connecting the target material and the particular region, characterized by further comprising:
an energy density setting means for setting energy strength of the laser beams to equal to or greater than 300 mJ, and setting density of the energy so as to fall within a predetermined range on the target material, wherein
a distance from an irradiation surface of the target material to the outflow window is set to ten or more times larger than a beam size on the surface of the target material.
